(19) United States
(12) Patent Application Publication
(10) Pub. No.: US 2020/0375628 A1
(43) Pub. Date: Dec. 3, 2020

(54) DYNAMIC EXTERNAL FIXATOR SYSTEM FOR SMALL JOINTS

(71) Applicant: Horizon IP Pte Ltd, Singapore (SG)

(72) Inventors: Tun Lin FOO, Singapore (SG); Amitabha LAHIRI, Singapore (SG); Eu Jin Andre CHEAH, Singapore (SG)

(21) Appl. No.: 16/884,066

(22) Filed: May 27, 2020

Related U.S. Application Data
(60) Provisional application No. 62/853,066, filed on May 27, 2019.

(51) Int. Cl. A61B 17/64; A61B 17/66

(52) U.S. Cl. CPC A61B 17/645; A61B 2017/564; A61B 17/66

(57) ABSTRACT

A method and system for a dynamic external fixator. The fixator system includes an elongated frame plate with apertures configured to accommodate bone fasteners to secure the elongated frame plate to a bone at an injured site. The fixator system includes primary and secondary mounts configured to accommodate rods to form a force vectoring subsystem that allows for early mobilization with minimal risk of complications, while at the same time ensuring minimal interference with the wearer's daily activities.

### CROSS-REFERENCE TO RELATED APPLICATION

This application claims the benefit of U.S. Provisional Application No. 62/853,066, filed on May 27, 2019, which is incorporated herein by reference in its entirety for all purposes.

### FIELD OF THE INVENTION

The present disclosure relates to external fixator systems. In particular, the present disclosure relates to compact dynamic external fixator systems utilizing a force vector approach to provide physical support to injured joints while minimizing interference on wearer's daily activities.

### BACKGROUND

External fixator systems are generally used in treatment of joint injuries. The injuries can include fractures, joint deformities, and soft tissue injuries. In particular, external fixator systems are primarily utilized in bone and joint injuries to prevent movement while bone and soft tissue healing take place. About the digits, a series of pins, connected by a frame, is surgically inserted into both sides of a phalangeal facture to support the affected bone or joint to facilitate tissue healing. The use of external fixators in areas with scarce skin or soft tissues is especially useful because they cause lesser disruption of the soft tissues and minimize obstruction of blood supply to the bones which is vital for healing. However, this prolonged fixture often results in stiffness and eventual disability due to inadequate active joint motion which helps in cartilage repair and regeneration.

Subsequent methods and devices have been developed to provide for sufficient fixation while allowing for certain degree of flexion during the treatment process. However, pin tract infections are prone to occur due to increased movement at the bone-pin interface. Secondary fracture displacements become more frequent as a result of increased load on the pins during movement. In addition, current systems are generally bulky and large, and hence uncomfortable for wear, as well as interfering with normal activities.

Therefore, based on the foregoing discussion, there is a desire to provide an improved method and system for a dynamic external fixator which is compact as well as utilizing a force vector approach to achieve desired fracture reduction and alignment, and still allow for early mobilization with minimal risk of complications, while at the same time ensuring minimal interference with the wearer's daily activities.

### SUMMARY

Embodiments generally relate to methods and devices for compact dynamic external fixators, such as external fixator systems which utilize a force vector approach to provide physical support to injured joints while maintaining a compact design for minimizing interference on wearer's daily activities.

In one embodiment, an external fixator includes an elongated frame plate having a frame body with first and second sides and top and bottom surfaces, apertures disposed along a length of the frame body. The apertures extend through the frame body from the top surface to the bottom surface and are configured to accommodate bone fasteners to secure the elongated frame plate. The external fixator further includes a primary mount configured to accommodate a primary rod which, when mounted onto the elongated frame member, extends beyond a second end of the frame plate. The external fixator further includes a secondary mount configured to accommodate a secondary rod, the secondary mount is disposed at about a first end of the frame body, the secondary rod, when mounted, extends transversely across the sides of the frame body.

In one embodiment, a method for supporting an injured joint includes coupling an elongated frame plate of a fixator system to a bone to form a fixated bone using bone fasteners through apertures on the elongated frame plate. The frame plate includes primary and secondary mounts configured to respectively accommodate primary and secondary rods. The method further includes mounting the primary rod to the primary mount of the frame plate, the primary rod extends beyond a second end of the elongated frame plate. The method further includes inserting an internal rod, along a transverse plane, into an adjacent bone next to the fixated bone, The primary rod and the internal rod forms a force vectoring subsystem of the fixator system. The method further includes coupling the internal rod to the primary rod by at least one vectoring connector to a cantilevered frame. The cantilevered frame is configured to minimize interference while enabling joint mobilization.

These and other advantages and features of the embodiments herein disclosed, will become apparent through reference to the following description and the accompanying drawings. Furthermore, it is to be understood that the features of the various embodiments described herein are not mutually exclusive and can exist in various combinations and permutations.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of various embodiments. In the following description, various embodiments of the present disclosure are described with reference to the following, in which:

FIGS. 1a-b show various embodiments of a dynamic external fixator;

FIGS. 2a-b show top and side views of an exemplary frame plate;

FIGS. 3a-b show side cross-sectional views of a side of a frame plate of an embodiment of a dynamic external fixator;

FIGS. 4a-c show top and side cross-sectional views of different configurations of the frames of a dynamic external fixator;

FIGS. 5a-b show side or top cross-sectional views of various primary frames in a first configuration of a dynamic external fixator;

FIG. 5c shows side cross-sectional views of exemplary modes of action of a primary frame in the first configuration;

FIG. 5d shows a side cross-sectional view of an exemplary primary frame in the first configuration;

FIGS. 6a-d show different applications of a first configuration of a dynamic external fixator;

FIGS. 7a-b show side cross-sectional views of force mechanisms in a second configuration of a dynamic external fixator; and

FIGS. 8a-m show an exemplary process of assembling a dynamic external fixator in a first or second configuration for treatment of an injured proximal interphalangeal joint (PIPJ).

### DETAILED DESCRIPTION

Embodiments described herein generally relate to methods and devices for compact dynamic external fixators. In particular, the dynamic external fixators are configured to utilize a force vector approach to provide physical support to injured joints through stability and fracture reduction, while allowing for early mobilization without risk of complications including infections. Further, a compact design is maintained, therefore minimizing interference on wearer's daily activities.

As discussed, external fixators are especially useful in fracture treatment of small bones in areas covered by scarce soft tissues and skin because they cause lesser disruption to the soft tissues and minimize obstruction of blood supply to the bones which is vital for healing. For example, external fixators are useful for applying to fractures in small bones such as phalanges in hands and feet.

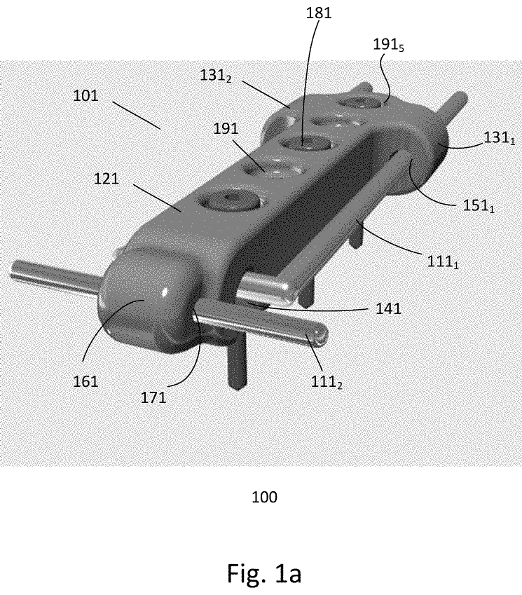

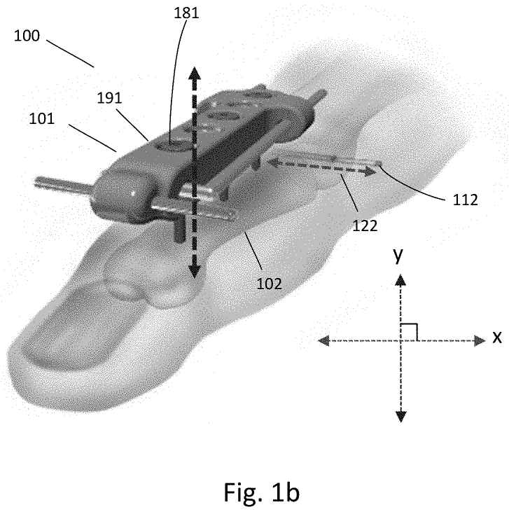

FIG. 1a shows an embodiment of external components of a dynamic external fixator 100 and FIG. 1b shows the dynamic external fixator 100 applied to a middle phalanx 102 of a human hand. The fixator may be referred to as a fixator system which includes both external components and internal components. External components do not touch the human bone while internal components do.

The external components of the dynamic external fixator system 100 include a frame plate 101. The frame plate or body is an elongated member. The elongated member is configured for mounting onto a bone. For example, the elongated member is mounted onto a bone by bone fasteners 181. The bone fasteners, since they contact a bone, are considered internal components of the fixator system. The elongated member is secured along a length of a bone, providing structural support for bones around an injured site.

External components of the fixator system may also include rods. For example, external rods are provided. The frame plate is configured to accommodate the external rods. For example, the frame plate includes frame mounts for accommodating the external rods. The external rods form frames configured to guide joint mobility around an injured site via a force vectoring subsystem or technique. The external rods, when fitted to the frame plate, do not directly contact a bone. Although the frame plate is configured to accommodate more than one rod, it is understood that depending on the application, only one external rod may be used.

The fixator system may include an internal rod 112, as shown in FIG. 1b. The internal rod, for example, is in direct contact with an adjacent bone to which the frame plate is attached. The internal rod belongs to internal components of the external fixator and is in direct contact with an adjacent bone 122. The internal rod, for example, is drilled transversely along a coronal plane or x-axis into the adjacent bone. The internal and external rods form the force vectoring subsystem of the fixator system.

Referring back to FIG. 1a, in one embodiment, the frame plate includes an elongated frame body 121 with first and second flanges 131₁₋₂. The flanges extend from first and second sides of the elongated frame body at or at about a second end of the frame body to form a T-shaped frame plate. The flanges may be disposed at the second end or recessed slightly from the second end. The frame plate includes a recess 141 on an underside surface towards a first end of the frame body. The first flange includes a first through hole 151₁ and the second flange includes a second through hole (not shown).

The recess and the first and second through holes collectively function as mounting points of the primary mount to support a first external rod 111₁. The primary mount assembles with a first external rod to form a primary frame. In one embodiment, the first external rod extends beyond the frame plate to increase a total length of the external fixator for bridging adjacent bones beneath the external fixator.

A collar head member 161 is disposed at the first end of the frame body. The collar head includes a third transverse through hole 171 which functions as a mounting point of the secondary mount for receiving a second external rod 111₂ in the transverse plane. The secondary mount engages with a second external rod to form a secondary frame. In one embodiment, the frame body is integrated with the flanges, recess and collar head. For example, the frame body with the flanges, collar head and recess is a single piece member.

At least one vectoring connector is provided. A vectoring connector is configured to connect an external rod to an internal rod to form the force vectoring subsystem or a part of the force vectoring subsystem. For example, the fixator system can have various configurations, depending on the application. The different configurations may include different setups of the internal and external rods. Depending on the configuration, one or more vectoring connectors are used to form the force vectoring subsystem.

In one embodiment, a vectoring connector is a flexible band which is configured to exert a distraction force of the vectoring subsystem. The flexible band, for example, may be a rubber band. The amount of force exerted can be adjusted by size and strength of the flexible band. In one embodiment, the flexible band may be a dental rubber band. Other types of flexible bands may also be useful.

The frame body includes a plurality of apertures 191 for accommodating or receiving the bone fasteners. The apertures are disposed along a longitudinal length of the frame body. For example, the apertures are disposed along a central axis of the frame body along the length direction. The apertures are openings which extend from a first or top surface of the frame body to a second or bottom surface. In one embodiment, the plurality of apertures can include any numbers of apertures such that the external fixator can be used as a generic device without customization. For example, the number of apertures should be sufficient to ensure a secure hold of a wide range of bones with different lengths. The frame body, for example, may include 5 apertures. Providing the frame body with other numbers of apertures may also be useful. The number of apertures may depend on the application. Alternatively, the external fixator can be customized so that the number of apertures is determined based on a bone length of a particular user or patient.

The plurality of apertures, in one embodiment, is spaced uniformly across an entire frame body length. For example, a last aperture 191₅ can be positioned between the two flanges. Each aperture is uniformly separated from adjacent epicenter by a diameter. In another embodiment, the apertures are distributed across the entire frame body length to achieve a better stability between fixated bone and the frame.

An internal diameter of each aperture is compatible for receiving bone fasteners such as pins or screws. For example, the screw can include cortical screws, cancellous screws or locking head screws. Any other types of screw or pin suitable for fastening into a bone at injured site can also be used.

In FIG. 1b, the apertures 191 are engaged with the bone fasteners, for example screws 181, for securing a bone, for example a middle phalanx 102. The apertures are used to direct exact positions to which the screws are inserted into the bone. For example, the screws are drilled vertically along a sagittal plane or y axis into the middle phalanx. In one embodiment, 3 screws are engaged with alternate apertures of the external fixator for an even support distribution across an entire length of the bone to ensure bone stabilization. The frame plate 101 together with the screws 181 form a stable construct with the bone, for example, the bone is now a fixated bone. The stable construct serves as a structural guide and support to the fixated bone. In one embodiment, depending on a type, extent, and position of injury, the number of apertures engaged with the bone fasteners are configured for providing a stable construct.

In one embodiment, there is a gap between the frame plate and the fixated bone. For example, the apertures are configured to receive screws which are configured to fix and retain the frame plate of the external fixator at a distance away from the bone. Screw heads once in contact with the apertures are prevented from further tightening and remain in a locked configuration with the frame plate without needing compression from plate-bone contact to form the stable construct. As a result of the contact, axial load of the screws is shared and distributed across the frame plate, to minimize the risk of screw loosening.

The apertures and the screws together form bone stabilization points so that the frame plate is tightly coupled to the fixated bone. For example, the stabilization points serve to secure one of the bones, for example the middle phalanx, at the injured site, to the external fixator. The frame plate is locked to the fixated bone and does not detach easily under disturbances. In addition, the frame plate also serves as a structural guide and support to the fixated bone.

In one embodiment, the bone stabilization points are distinct from distraction points formed between the coupled external and internal rods. The distraction points, support mobility of the fixated bone along a joint at the injured site. Joint mobility is needed for movements such as flexion and/or extension of an injured finger, which involves moving a phalanx along an anatomical center of rotation at a joint.

Maintenance of joint mobility is crucial during treatment of bone injuries caused by physical trauma, or diseases such as Arthritis. For example, the distraction points enable a sustained mobility to a joint contracture site and therefore aids in restoring a full motion range of an initially motion-restricted joint. In Dupuytren's contracture, a contracture is caused by underlying diseased skin tissue forming a thick cord which restricts finger movements. The external fixator alleviates and improves the contracture by allowing soft tissues of the diseased area to elongate progressively via the distraction points which promote progressive stretching of the diseased cords and skin and therefore facilitate rehabilitation of the finger and joint functions.

In one embodiment, the distraction points are controlled by the at least one connector used to connect the external and internal rods. The connectors connect free ends or coupling ends of the external rods to free ends or coupling ends of the internal rod. Various types of connectors that are elastic and flexible can be used. In one embodiment, the connectors are dental rubber bands.

In one embodiment, the at least one of the connectors controls a fixated bone to move along an anatomical center of rotation along an affected joint by pulling on the frame plate. For example, during flexion of an injured finger, the coupling ends of the internal rod act as anchoring points of the external fixator while the connectors exert a downward force on the frame plate via pulling of the external rod. A bone fixated to the external fixator is in turn guided along a same direction as the frame plate, thereby facilitating an anatomical rotation of the fixated bone along an affected joint.

During joint mobility, the at least one connector (e.g., elastic component) of the construct provides load support to the injured joint. This offloads pressure from the joint and prevents further insult to soft tissues about the joint. Overall, a more effective healing process is achieved.

In another embodiment, the at least one connector directs bone displacement required for corrective joint alignment and fracture reduction. A bone fracture, for example a phalanx fracture, may lead to joint dislocation and migration. Application of the external fixator to the affected site aids in reducing the dislocated joint back to an original anatomical alignment by exerting at least one force vector via the at least one connector to direct desired bone displacement.

The fixator system is designed with flexibility to accommodate different applications, such as different injuries and different size bones. For example, depending on the injuries, the external and internal rods generate the desired force vectoring system while the frame body may be securely mounted onto a bone by using the appropriate apertures based on size of the bone.

The frame plate should be formed from a sufficiently rigid material to provide support to the bone as well as serving as a fixator system to support the external rods and bone fasteners. For example, the frame plate may be formed of a resin. Other types of rigid materials, such as metals, may also be useful. The frame plate may be formed as a single piece (integrated) unit. For example, the frame plate may be designed using a computed aided design (CAD) system. Various processes may be employed to form the frame plate. For example, 3D printing or injection molding may be used to form the frame plate.

As for the apertures, they may be designed with bore according to the design. As a guide, for a resin frame, the diameter should not exceed ⅓ of the width or height, whichever is smaller. For example, assume a cross-section of the frame is 5×6 mm, the aperture should be limited to about 1.6 mm. In the case of a metal frame, the aperture may be limited to ½ of the narrower of the frame width or height. The diameter of the apertures may be adjusted at time of use, based on the size of the bone fasteners used. For example, apertures may be formed with an initial diameter which may be enlarged subsequently. For example, a drill or tap may be used to enlarge the diameter of the apertures at a later stage. However, the final diameter should be limited by the upper diameter limits.

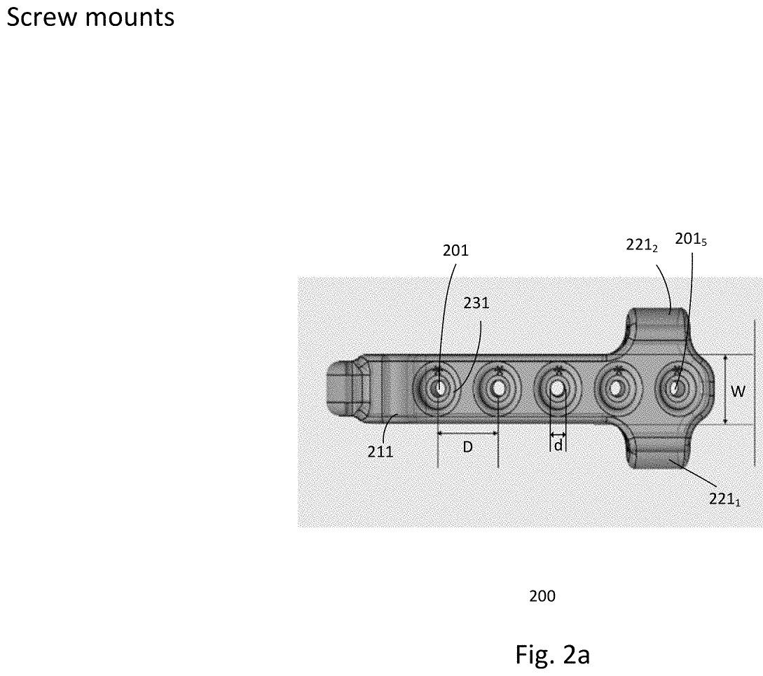

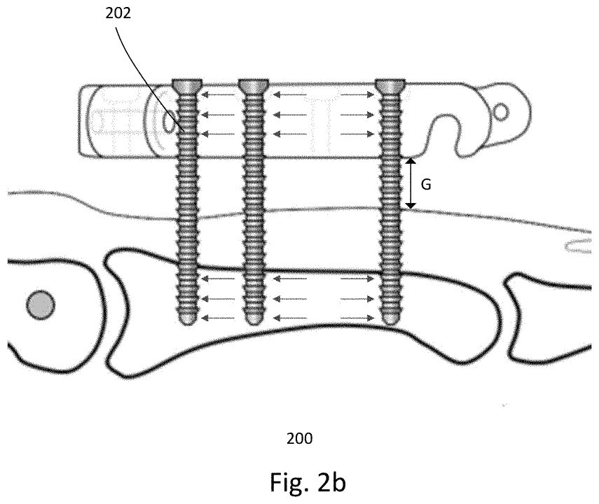

FIG. 2a shows a top planar view of an exemplary frame plate 200. The frame plate includes a plurality of apertures 201 disposed along a longitudinal length of the frame body 211. The apertures are configured for accommodating the bone fasteners. As shown, in one embodiment, the apertures include five apertures. Alternatively, any other numbers of apertures can be used. The apertures are openings which extend from a first surface or top surface of the frame body to a second surface or bottom surface.

The plurality of apertures, in one embodiment, is spaced across an entire frame body length. For example, a last aperture 201₅ can be positioned between the two flanges 221₁₋₂. In another embodiment, the apertures are uniformly spaced across the entire frame body length and each aperture is uniformly separated from adjacent epicenter by a diameter D. In one embodiment, D equals to 4 mm.

Each internal diameter of the apertures is compatible for receiving any surgeons' choice of pin or screw types. For example, the screw can include cortical screws, cancellous screws and locking head screws. Any other types of screw or pin suitable for fastening into a bone at injured site can also be used. In one embodiment, the internal diameter, d, of each aperture is 1.25 mm.

As shown in FIG. 2b, the apertures are engaged with bone fasteners 202, for example screws, for securing a bone, for example a middle phalanx. The apertures are used to direct exact positions to which the screws are inserted into the bone. For example, the screws are drilled vertically along a sagittal plane into the middle phalanx. As shown, in one embodiment, not all apertures have to be engaged. As long as the screws are engaged with apertures at positions that provide for an even support distribution across an entire length of the bone to ensure bone stabilization.

Returning back to FIG. 2a, the apertures, in one embodiment, include grooves 231 suitable for receiving threaded pins or screws. The screws can be partially or fully threaded and can include unthreaded or threaded screw heads. In one embodiment, the apertures are configured to receive screws with threaded screw heads to form a fixed angle construct. For example, the groove of each aperture is configured to mate with one threaded screw head. The mating between the grooved apertures and the threaded screw heads of the screws maintains the frame plate and the bone in a locked configuration. As shown in FIG. 2b, through locked engagement between the frame plate and the screws, axial force of the screws is transmitted over a longer plate length for an even load distribution. This prevents screw loosening, secondary loss of reduction, and material fatigue.

In one embodiment, as shown in FIG. 2b, the frame plate together with the screws form a stable construct with the attached bone with a gap G formed between the frame plate and the attached bone. For example, the apertures are configured to receive screws which are configured to fix and retain the frame plate of the external fixator at a distance away from the bone. Screw heads, once in contact with the apertures, are prevented from further tightening and remain in a locked configuration with the frame plate without needing compression from plate-bone contact to form the stable construct. Unlike a conventional external fixator which ensures fixator-bone construct stability through tight compression between a frame plate and the bone, the locked configuration between the mated grooved apertures and the threaded screw heads provides for angular stability without requiring plate-bone contact. This allows for uninterrupted blood supply to the bone without any compression on periosteum, and therefore facilitates faster bone healing. In one embodiment, the gap G allows for sufficient space for possible soft tissue swelling. For example, even during swelling of the soft tissue around the fixated bone, the frame plate is fixated at a height such that the frame plate is not in contact with the surrounding skin of the injured area.

In another embodiment, the apertures and the screws together form bone stabilization points so that the frame plate is tightly coupled to the fixated bone. For example, the stabilization points serve to secure one of the bones, such as the middle phalanx, at the injured site, to the external fixator. The frame plate is locked to the fixated bone and does not detach easily under disturbances. In addition, the frame plate also serves as a structural guide and support to the fixated bone. In one embodiment, the bone stabilization points are distinct from distraction points between the coupled external and internal rods.

The frame body, in one embodiment, has a width that is wide enough to accommodate grooved apertures suitable for receiving threaded pins or screws. For example, as indicated in FIG. 2a, the frame body has to be wide enough to hold the grooved apertures which include an internal diameter that is wide enough for receiving any of the surgeons' choices of pin or screw types. For example, the frame body may have a width W of about 5 mm in order to accommodate grooved apertures having an internal diameter of about 1.25 mm. Other frame body and grooved aperture dimensions may also be useful. As discussed, the frame plate serves as a structural guide and support to bones around the injured site.

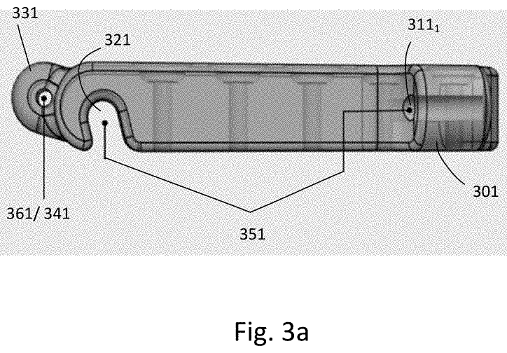

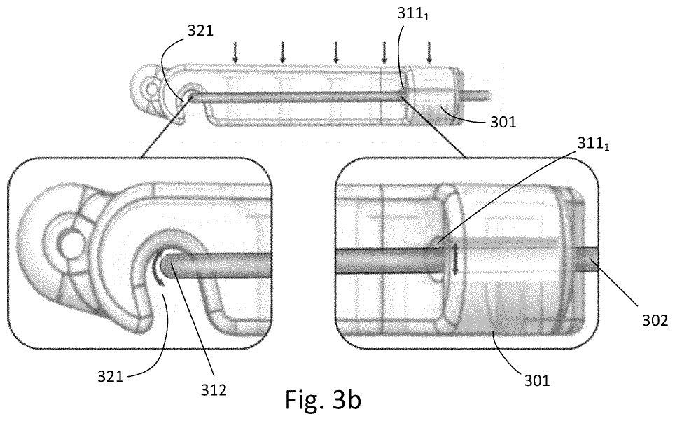

FIGS. 3a-b show side cross-sectional views of a side of a frame plate of an embodiment of a dynamic external fixator. In one embodiment, the frame plate includes a primary mount 351 and a secondary mount 361. The primary mount is configured to engage one of the external rods, for example a first external rod, to facilitate first external coupling points. The secondary mount is configured to engage another of the external rods, for example a second external rod, to facilitate second external coupling points.

In one embodiment, as seen in FIG. 3a which illustrates one side of the frame plate, a first flange 301 of the frame plate is disposed on a side of the frame body. Even though it is not shown, it is to be understood that a second flange is disposed on another side of the frame body. The first flange includes a first through hole 311₁ and the second flange includes a second through hole (not shown). A recess 321 is formed on an underside surface of a first end of the frame body. The recess and the first and second through holes collectively function as mounting points of the primary mount 351 to support a first external rod.

As for the secondary mount, a third through hole 341 is transversely disposed in a collar head member 331 integrally formed at the first end of the frame body. The third through hole functions as a mounting point of the secondary mount 361 for receiving a second external rod in a transverse plane.

A primary mount engaged with one external rod forms a primary frame. The primary frame may be part of a first configuration or a second configuration of an external fixator. In one embodiment, the primary frame is configured to provide load support and joint mobility at an injured site. In another embodiment, when the injured site includes a fractured and/or dislocated joint, the primary frame also controls a first force vector for joint alignment and fracture reduction.

A secondary frame mount engaged with another external rod forms a secondary frame. The secondary frame may be part of a second configuration of an external fixator. The secondary frame mount controls a second force vector which is summed up with the first force vector from the primary frame to generate a net bone displacement. When the primary frame in a first configuration is not sufficient to provide smooth joint mobility and/or complete fracture reduction, the secondary frame is assembled to form a second configuration. The second configuration is used to generate a desired net bone displacement for more complete fracture reduction.

In one embodiment, the first external rod is a U-shaped rod. The U-shaped rod is configured to engage the primary mount. As shown from one side of the primary mount in FIG. 3b, a first end 302 of the U-shaped rod is inserted into the first through hole 311₁ of the first flange 301 of the frame plate. Even though it is not shown in the figure, it is to be understood that the first and second free ends of the U-shaped rod are simultaneously inserted into first and second (not shown) through holes of the first and second flanges. A central limb 312 of the U-shaped rod is then slotted into the recess 321 formed on a bottom surface of a first end of the frame body so that the central limb and the two free ends of the rod are aligned along a same plane. The first and second through holes and the recess together facilitate as mounting points of the primary mount to support the U-shaped rod in a horizontal plane. Alternatively, any other types of wires suitable for engaging the primary mount may also be used.

In one embodiment, the primary mount is configured to engage a first external rod having a diameter of less than or equal to 1.4 mm. In FIG. 3b, a sloppy primary mount is used in another embodiment. The sloppy mount accommodates for interferences caused by movements of the external rod around the mounting points. This minimizes transmission of vibrations to the bone fasteners which can otherwise lead to loosening of the bone fasteners. For example, the primary mount includes a recess and first and second through holes having diameters configured to loosely fit the external rod during engagement. The loose fit of the external rod to the sloppy primary mount reduces transmission of vibrations to the bone fasteners by reducing a surface area of contact between the external rod and undersides of the recess and the through holes.

In one embodiment, the primary mount includes first and second through holes having an internal diameter of 1.5 mm and a recess having a 2.0 mm wide diameter so as to loosely engage an external rod having a diameter of less than or equal to 1.4 mm. Other configurations of the primary mount for fitting an external rod having other diameters may also be used.

In one embodiment, the third through hole of the secondary mount has a diameter configured to receive a second external rod having a dimeter less than or equal to 1.1 mm. For example, the diameter of the third through hole is 1.1 mm. Alternatively, a third though hole having other diameters configured to receive a second external rod having other diameters may also be provided.

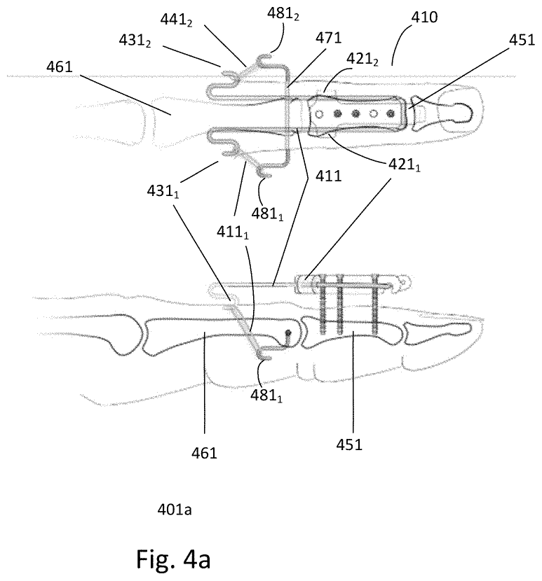

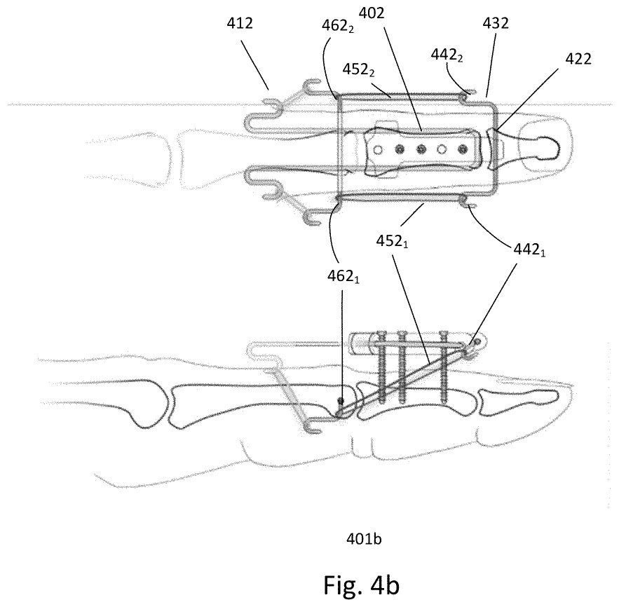

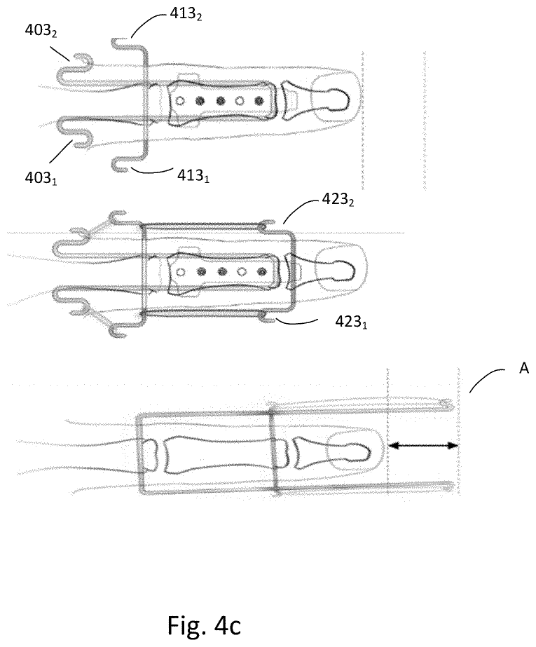

FIGS. 4a-c show top and side cross-sectional views of different configurations of the frames of a dynamic external fixator. In FIG. 4a, the U-shaped rod 411 is configured to engage the primary mount to form a primary frame in a first configuration 401a. The first configuration does not include engaging the secondary mount. As shown, in one embodiment, the primary frame is a bilateral primary frame 410. The bilateral primary frame will be useful for providing support to both sides, for example both left and right sides, of an injured site. The free ends of the U-shaped rod extend out respectively from each side, for example the first 421₁ and second 421₂ flanges, of the frame plate. For example, both lengths of the first and second free ends 431₁₋₂ extend out respectively from the first and second through holes.

Alternatively, the frame is a unilateral primary frame. The unilateral primary frame will be useful for treating a unilateral injured side, for example, a single-sided ligament insufficiency caused by Arthritis, by providing ligament support at the unilateral injured side. The primary mount engages with the U-shaped rod such that only one free end of the U-shaped rod is extending out from a side, for example, one of the flanges, of the frame body. Only a length of one of the free ends is extending out from one of the through holes. A length of another of the free ends is removed and does not extend beyond another of the through hole. The free end is constrained within the frame for angular stability. Using either the first or second free end of the U-shaped rod to form a unilateral primary frame depends on which side of the affected site is injured. For example, a unilateral primary frame includes one free end extending at a same side as the unilateral injured side.

The extended free ends of the U-shaped rod are configured to serve as coupling points for at least one first connector to connect the fixated bone. In one embodiment, in FIG. 4a, a bilateral primary frame includes both extended free ends 431₁₋₂ of the U-shaped rod which is configured to serve as coupling points for two first connectors 441₁₋₂ to connect the fixated bone 451, for example a middle phalanx, to an adjacent bone 461, for example a proximal phalanx. The two first connectors connect the U-shaped rod to an internal rod 471 at both sides, for example left and right sides, of the injured side. A length of each of two free ends 48112 of the internal rod is configured to remain protruding out from skin at an inserted site to receive the first and second connectors for connection to the first and second extended free ends of the U-shaped rod.

In another embodiment, a unilateral primary frame includes one extended free end of the U-shaped rod which is configured to serve as a coupling point for a first connector to connect the fixated bone, for example a middle phalanx, to an adjacent bone, for example a proximal phalanx. The first connector connects the U-shaped rod to an internal rod at the unilateral injured side. For example, a length of one free end of the internal rod is configured to remain protruding out from skin at an inserted site and the protrusion is at a same side as the extended free end of the U-shaped rod. As for another free end of the internal rod which is not required for connecting to the U-shaped rod, the free end is maintained at a length configured to minimize interference with the patient's daily activities.

In FIG. 4b, both the primary and secondary mounts are engaged to external rods to form a second configuration 401b. The second configuration includes a primary mount engaged to a first external rod 402 to form a primary frame 412 in a manner similar to the first configuration as discussed above. The primary frame can be unilateral or bilateral. The second configuration further includes a secondary mount engaged to a second external rod 422 to form a secondary frame 432.

In one embodiment, the secondary mount includes a collar head member integrally formed at a first end of the frame body. The collar head includes a third transverse through hole for receiving a second external rod in the transverse plane. In one embodiment, the third through hole has a diameter configured to receive a second external rod having a diameter less than or equal to 1.1 mm. For example, the diameter of the third through hole is 1.1 mm. Alternatively, a third though hole having other diameters configured to receive a second external rod having other diameters may also be provided.

The second frame mount includes inserting the second external rod into the third through hole so that each of the two free ends of the second external rod is extending out from opposing openings of the third through hole.

The extended free ends of the second external rod is configured to serve as coupling points for at least one second connector to connect the fixated bone and the adjacent bone which are already in connection via the primary frame. In one embodiment, two second connectors 452₁₋₂ connect both extended free ends 442₁₋₂ of the second external rod to the free ends 462₁₋₂ of the internal rod at both sides, for example left and right sides, of the injured side.

The external rods together with the connected internal rod, in one embodiment, form a cantilevered configuration as shown in FIG. 4c. For example, the two free ends 403₁₋₂ of the first external rod in the primary frame are bent in first and second directions to form S-shaped overhangs. Similarly, the two free ends 423₁₋₂ of the second external rod in the secondary frame are bent in first and second directions to form S-shaped overhangs. The same bending also applies for the internal rod 413₁₋₂ inserted into the adjacent bone. The cantilevered design of the rods minimizes an overall dimension of the dynamic external fixator and therefore reduces interference of the fixator on a patent's daily activities while providing treatment. For example, unlike an external fixator of a prior art A as shown, the overall dimension of the dynamic external fixator is not longer than a finger length. This reduces obstruction to finger activities, and hence provides improved comfort to the patient while retaining functionality throughout treatment process.

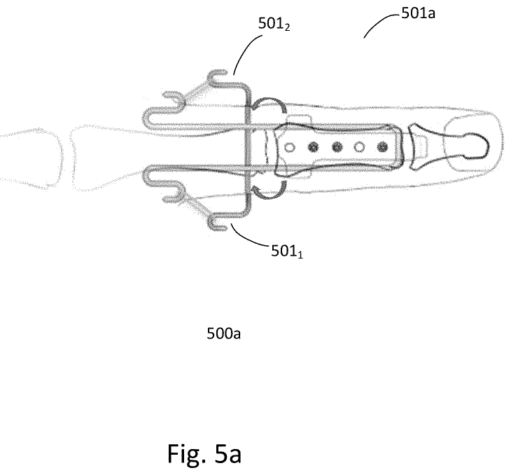

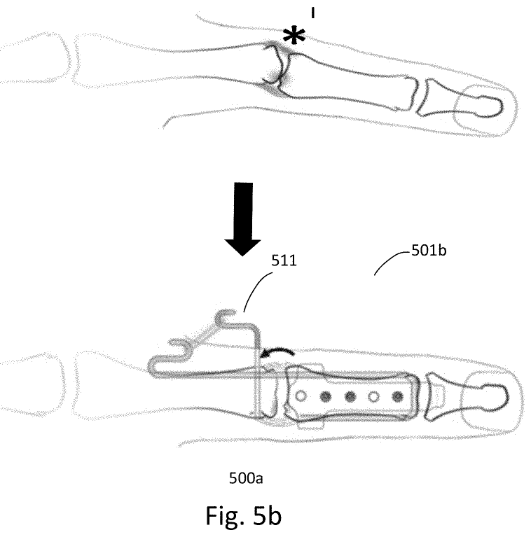

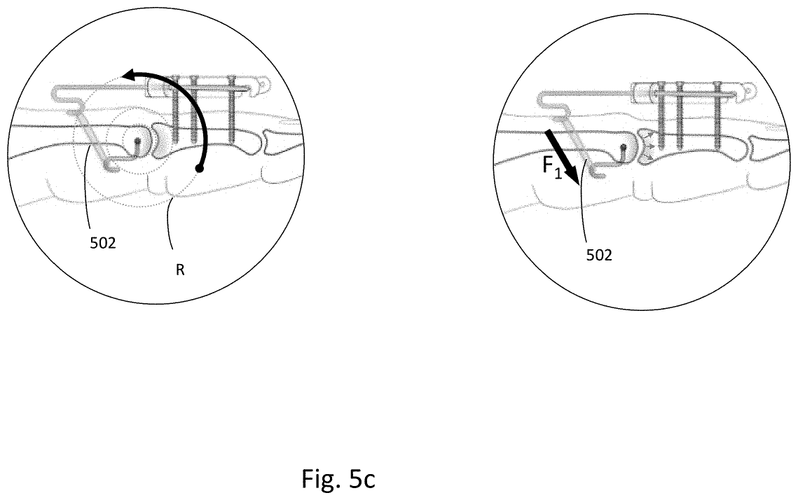

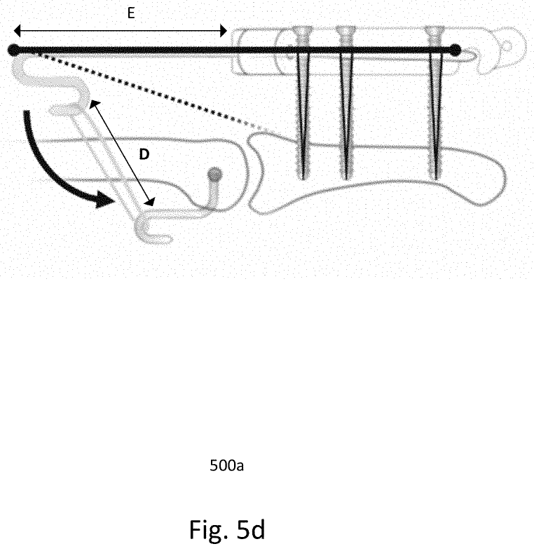

FIGS. 5a-b show side or top cross-sectional views of various primary frames in a first configuration of a dynamic external fixator. As discussed, the first configuration includes engaging a first external rod with the primary mount to form a primary frame. The first configuration does not include engaging the secondary mount.

The primary frame can be either bilateral or unilateral. The bilateral primary frame is useful for providing support to both sides, for example both left and right sides, of an injured site. The unilateral primary frame will be useful for treating a unilateral injured side, for example, a single-sided ligament insufficiency caused by Arthritis, by providing ligament support at the unilateral injured side.

In one embodiment, in FIG. 5a, a first configuration 500a includes a bilateral primary frame 501a. The free ends of the U-shaped rod extend out respectively from each side, for example the first and second flanges, of the frame body to form the bilateral primary frame. As shown, both lengths of the first and second free ends 501₁₋₂ are extended out respectively from the first and second through holes.

Alternatively, in FIG. 5b, the first configuration 500a includes a unilateral primary frame 501b. The primary mount engages with the U-shaped rod such that only one free end 511 of the U-shaped rod is extending out from a side, for example, one of the flanges, of the frame body. Only a length of one of the free ends is extending out from one of the through holes. A length of another of the free ends is removed and does not extend beyond another of the through hole. Using either the first or second free end of the U-shaped rod to form a unilateral primary frame depends on which side I of the affected site is injured. For example, as seen in FIG. 5b, a unilateral primary frame includes one free end extending at a same side as the unilateral injured side.

FIG. 5c shows side cross-sectional views of exemplary modes of action of a primary frame in the first configuration 500a. The primary frame supports joint mobility via the at least one first connector. The at least one first connector controls distraction points for supporting joint mobility at the injured site. Joint mobility is needed for movements such as flexion and/or extension of an injured finger, which involves moving a phalanx an anatomical center of rotation at a joint. Maintenance of joint mobility is crucial during treatment of bone injuries caused by physical trauma, or diseases such as Arthritis. For example, controlling the distraction points guides a sustained mobility to a joint contracture site, and therefore aids in restoring a full motion range of an initially motion-restricted joint. In Dupuytren's contracture, a contracture is caused by underlying diseased skin tissue forming a thick cord which restricts finger movements. The contracture can be alleviated and improved if the soft tissues of the diseased area, for example the diseased cords and skin, can elongate progressively. Controlling of the distraction points guides progressive stretching of the diseased cords and skin, and therefore facilitate rehabilitation of the finger and joint functions.

In one embodiment, as shown in FIG. 5c, at least one first connector 502 controls a phalanx to move along an anatomical center of rotation R along a joint by pulling on one side of the frame body via the external rod. It is to be understood that even though only one side is shown, another first connector on another side also functions simultaneously in the same manner. For example, in a bilateral primary frame, the two first connectors control a phalanx to move along an anatomical center of rotation along a joint by pulling on both sides of the frame body via the external rod.

During joint mobility, the at least one first connector 502 facilitates load support of the joint. As shown in FIG. 5c, a load L on the joint is distributed across the external fixator through the connector. This offloads pressure from the joint and prevents further insult to soft tissues about the joint. Overall, a more effective healing process is achieved around the injured site.

For an injury involving a fractured and/or dislocated joint, at least one first connector also serves to provide at least one first force vector for fracture reduction and joint alignment. For example, as shown in FIG. 5c, one first connector provides one force vector F1 at one side of the injured side. Even though only one side is shown, it is to be understood that if there is another first connector on another side, the another first connector also functions simultaneously in the same manner.

The at least one first force vector generates a first displacement of the fixated bone along the affected joint. Exertion of the first force vector on the frame plate by the first connector causes the bone fixed to the frame plane to be displaced along a same direction as the frame plate. This realigns the fractured and/or dislocated joint back to an original anatomical alignment.

FIG. 5d shows a side cross-sectional view of an exemplary primary frame in the first configuration 500a. In one embodiment, an extent of rotation of a phalanx along an anatomical center of rotation at a joint is controlled by varying a length E of the external rod. For example, a longer length increases the connecting distance D between the coupling ends of the external rod and the internal rod, and therefore permits a wider degree of rotation about the joint. In one embodiment, the length of the external rod is determined by modifying an extent of extension of the free ends of the external rod.

Varying the length of the external rod also changes a magnitude and direction of the first force vector. In one embodiment, a length of the external rod can be used such that it is configured to achieve complete closure at the fracture and/or dislocated joint site. For example, a length of the free ends is configured to generate a first force vector with a desired magnitude and direction for fracture reduction and/or joint alignment. A major joint dislocation may require a longer external rod for a wider bone displacement about the affected joint.

The magnitude of the first force vector can also be independently controlled by the at least one first connector. Varying a tension of the at least one first connector can be used to control a first displacement magnitude. For example, one first connector with greater tension generates a greater first displacement. In one embodiment, dental rubber bands are used as the first connector. Any other types of connectors having different tension properties can also be used.

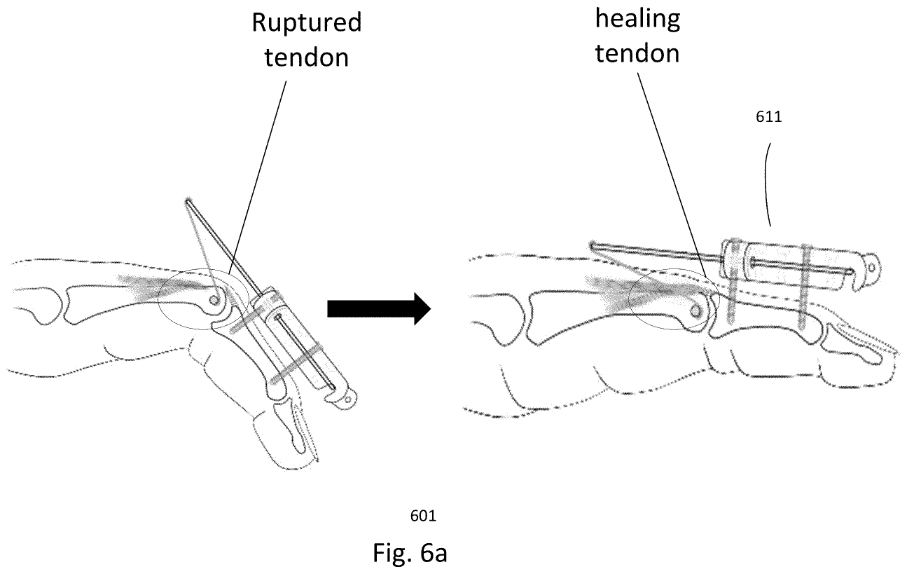

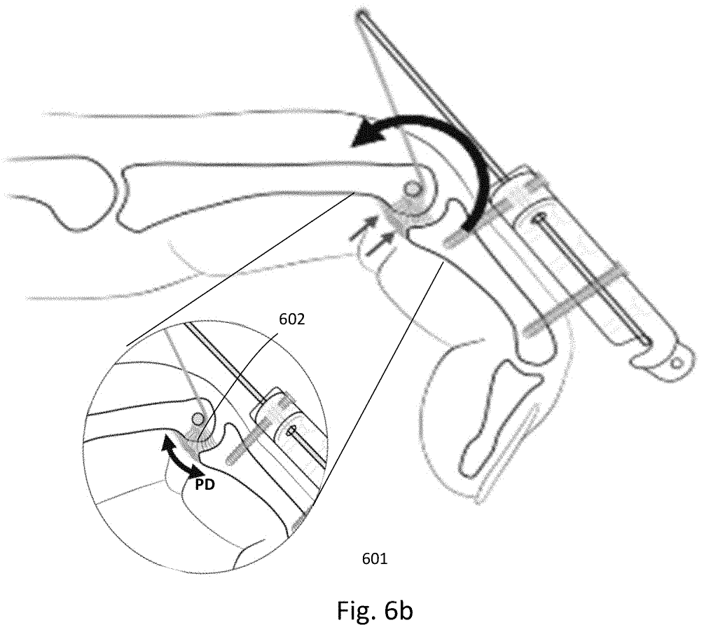

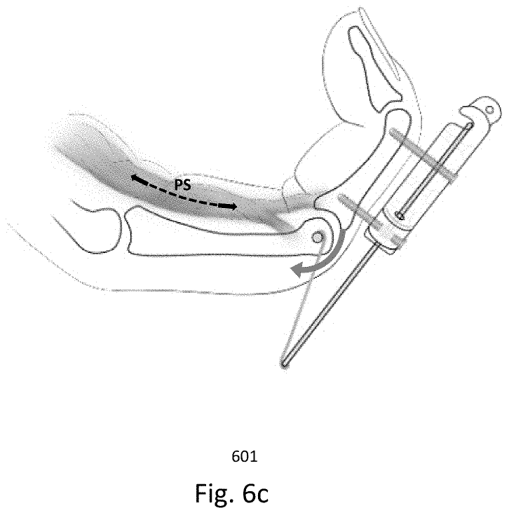

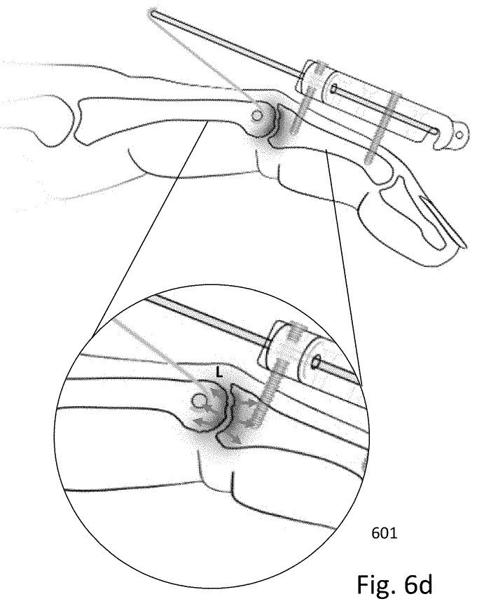

FIGS. 6a-d show different applications of a first configuration of a dynamic external fixator. Referring to FIG. 6a, a first configuration 601 is applied for treatment of tendon insufficiency at a proximal interphalangeal joint (PIPJ) region, whereby an impaired or ruptured tendon is unable to support straightening of an affected finger. The primary frame 611 provides support to maintain the affected finger in a straight position while tendon heals by secondary intention or following surgical reconstruction. In addition, the primary frame also supports PIPJ mobility while protecting the healing tendon.

A first configuration is also useful in treating joint contractures that commonly occur following physical trauma. In FIG. 6b, the first configuration 601 enables progressive distraction, in directions indicated by the arrow PD, of contracted soft tissues 602 along an injured joint, and thereby aids in restoring joint mobility.

Similarly, such an application can also be used in conditions such as Dupuytren's Contracture. In Dupuytren's contracture, a contracture is caused by underlying diseased skin tissue forming a thick cord which restricts finger movements. The first configuration 601 guides progressive stretching, in directions indicated by the arrow PS, of diseased cords and skin as illustrated in FIG. 6c. This facilitates rehabilitation of the finger and joint functions.

The primary frame of the first configuration 601 also facilitates load support of the injured joint during joint mobility. As indicated by arrows illustrated in FIG. 6d, during joint movement, a load L on an injured joint is neutralized by distribution across the frame plate through the primary frame and the bone fasteners. Joint injury can be caused by physical trauma or diseases such as degenerative or inflammatory Arthritis. Pressure is offloaded from the joint and this prevents further insult to soft tissues about the joint. Overall, a more effective healing process is achieved around the injured site.

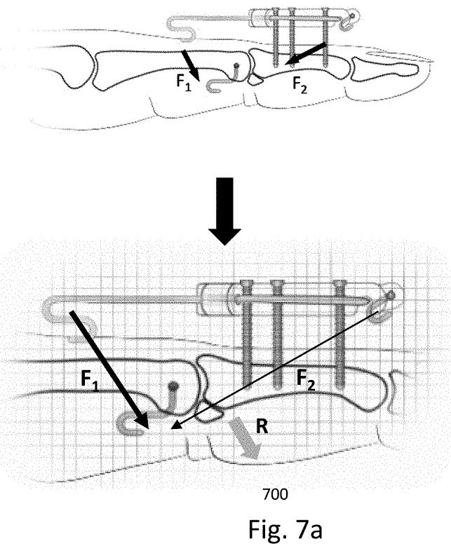

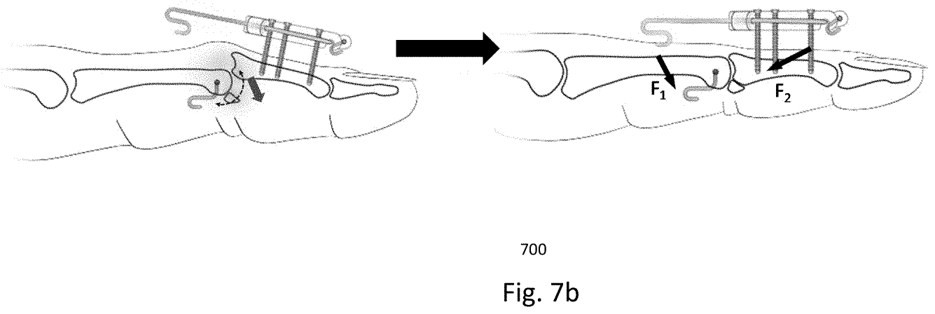

FIGS. 7a-b show side cross-sectional views of force mechanisms in a second configuration 700 of a dynamic external fixator. The second configuration includes a primary mount engaged to a first external rod to form a primary frame in a manner similar to the first configuration as discussed above. The primary frame can be unilateral or bilateral. The second configuration further includes a secondary mount engaged to a second external rod to form a secondary frame. In FIG. 7a, the secondary frame provides a second force vector F2, in addition to the first force vector F1, to generate a net displacement R of the fixated bone for a better fracture reduction result.

In one embodiment, a second configuration can be applied instead when the first configuration does not provide sufficient fracture reduction for proper joint realignment or smooth joint mobility. For example, a first force vector applied by a primary frame of a first configuration does not generate a desired direction of displacement required for a complete fracture reduction. In this case, in addition to the primary frame, a secondary frame can be installed to form a second configuration. The secondary frame applies a second force vector at a direction distinct from the first force vector. As a result of a summation of the first and second force vectors, a net displacement in the desired direction is generated. The direction of the net displacement is distinct from the first and second force vectors.

A direction and magnitude of the net displacement can be controlled to generate a desired displacement for maintaining or improving fracture reduction and joint congruity. In one embodiment, the direction and magnitude of the net displacement are determined by the first and second connectors. Varying tension of the first and/or second connectors can be used to control the net displacement. Generally, a net displacement is biased towards a side having the connectors with greater tension. For example, a connector with greater tension generates a greater force vector. A net displacement will be biased towards a first connector with greater tension. Depending on a magnitude and direction of force vector required, different types of connectors with different tension properties can be used for the first and second connectors.

In FIG. 7b, a second configuration 700 is applied to treat a proximal interphalangeal joint (PIPJ) dislocation caused by a fracture at base of a middle phalanx. Primary and secondary frames of the second configuration respectively exert first and second force vectors to generate a net displacement, for example in a downwards direction, to guide the dislocated joint back to an anatomical position. As discussed, a desired direction of the net displacement can be controlled by adjusting tension of the first and/or second connectors to achieve a more complete fracture reduction and smooth joint mobility.

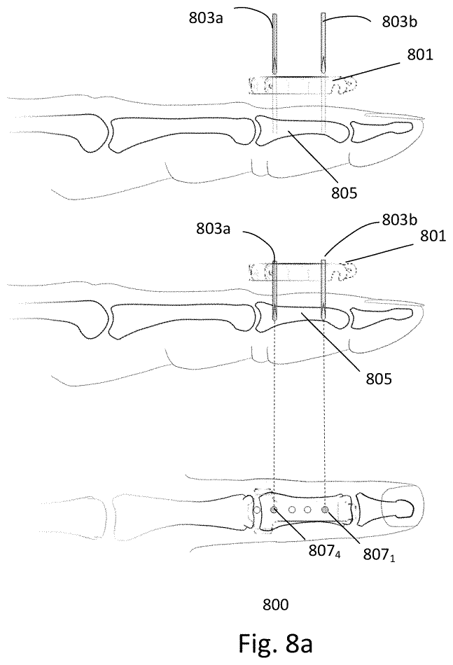

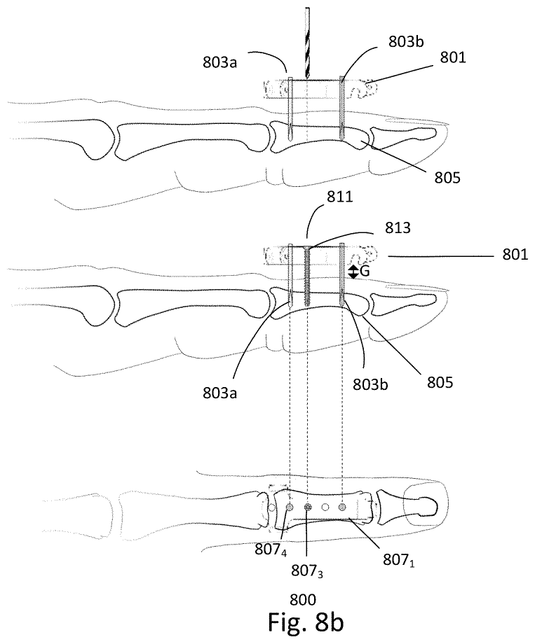

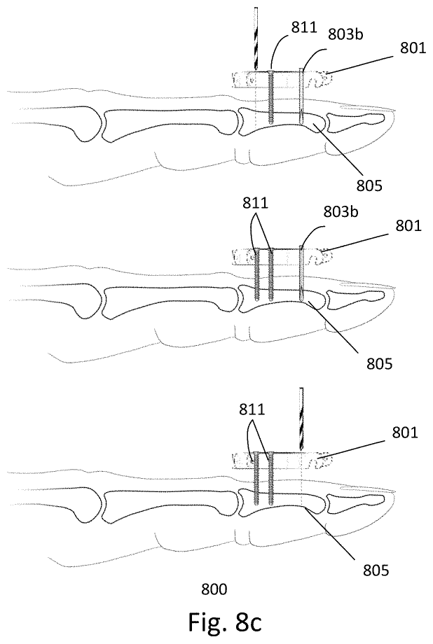

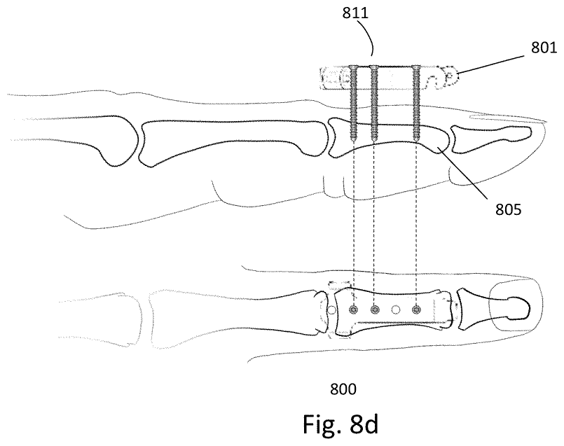

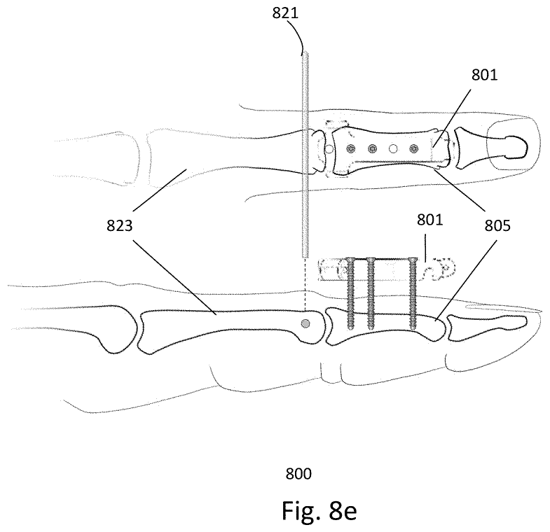

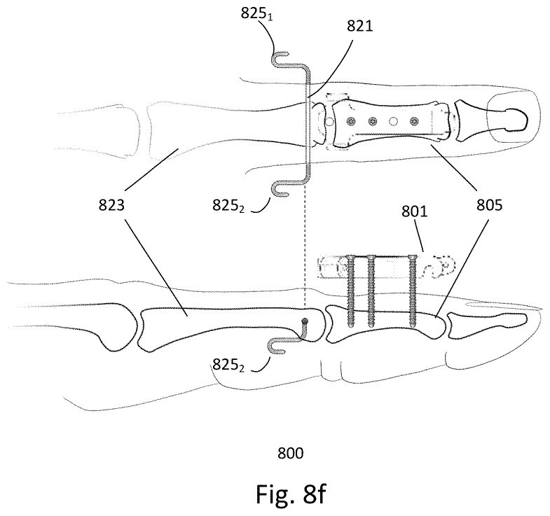

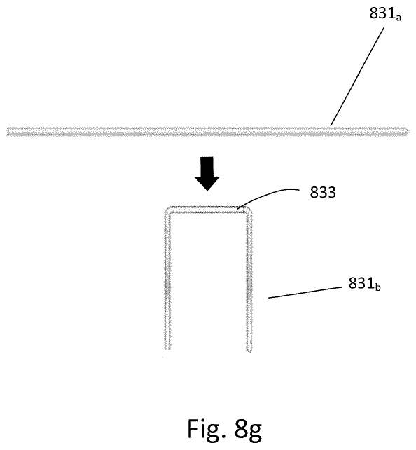

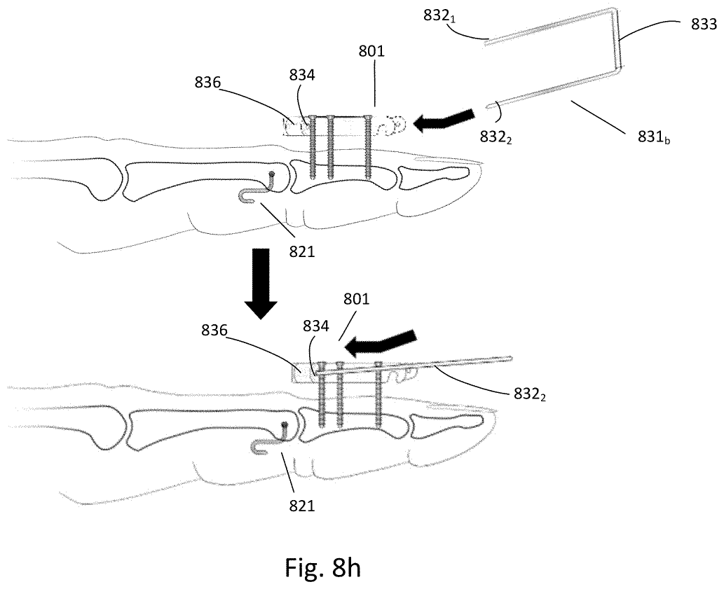

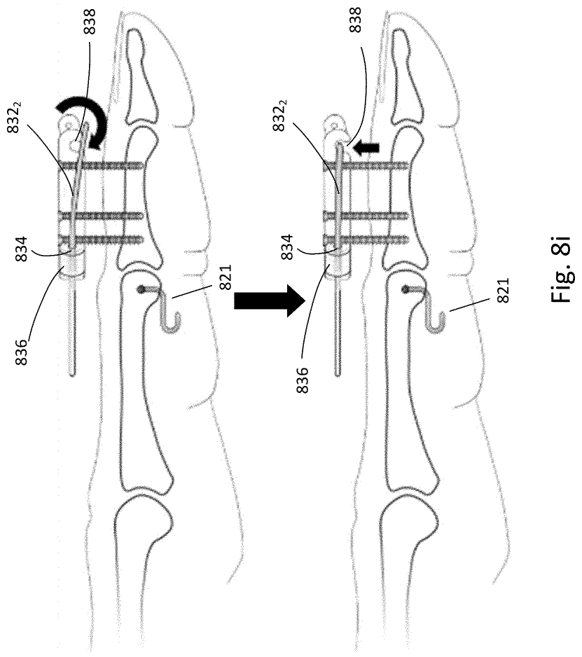

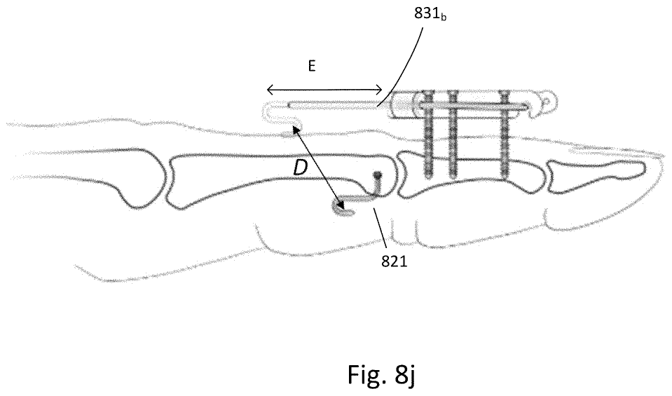

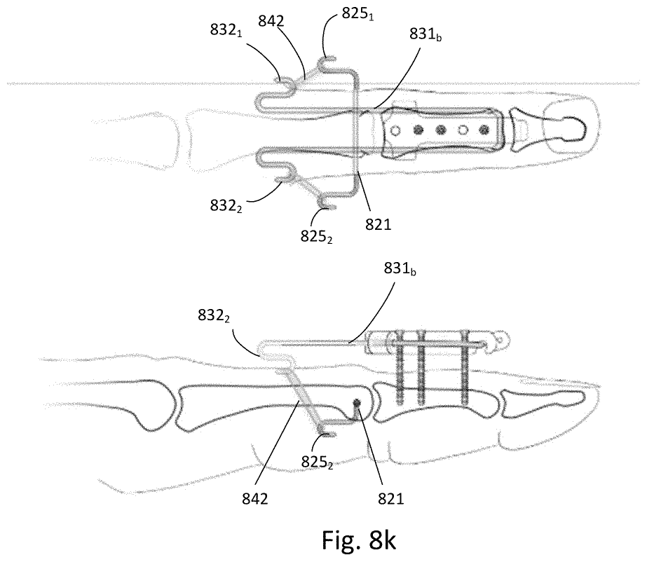

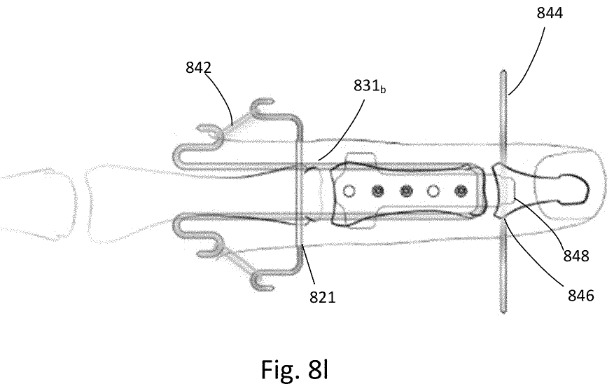

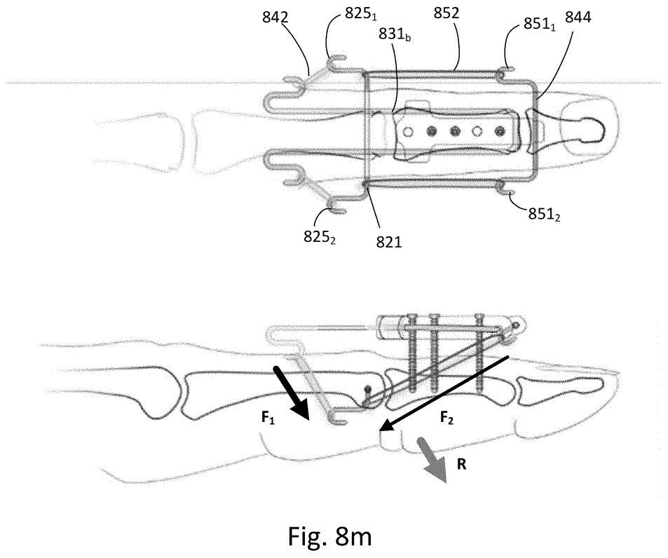

FIGS. 8a-m show an exemplary process 800 of assembling a dynamic external fixator in a first or second configuration for treatment of an injured proximal interphalangeal joint (PIPJ). As discussed, a dynamic external fixator can be effectively applied to treat injuries at small bones, in particular phalanxes at hands or feet. For example, an injured joint, such as a PIPJ, between the middle and proximal phalanxes.

Prior to application of the dynamic external fixator, images of surrounding injured area may be taken to determine an exact location and morphology of injured tissues and/or fracture. Any imaging techniques such as radiography, computed tomography (CT), and magnetic resonance imaging (MRI), may be utilized to provide support for preoperative surgical planning and/or guidance. Pre-operative planning may include determining skin and/or bone incision locations for application of external fixators and bone fasteners, the type of configuration of external fixator to be applied, the type and number of bone fasteners or screws, as well as lengths of the external and internal rods. It may also be useful to consider other factors that may affect the treatment process.

Referring to FIG. 8a, once pre-operative planning is completed and prior to actual fixation of the frame plate to a middle phalanx of the injured area, a frame plate 801 of the external fixator is orientated against a surface of the injured site. During the procedure, it may be useful to provide markings of entry points, which had been decided pre-operatively. In one embodiment, as shown in FIG. 8a, two wires 803a and 803b are disposed at opposing edges of the middle bone 805 which correspond to positions of first and fourth apertures 807₁ and 807₄, located on the frame plate of the external fixator. The two wires allow the frame plate to be orientated at a determined position before actual application of the external fixator to the middle phalanx. The wires are inserted perpendicularly to a longitudinal axis of the middle bone. The wires may include Kirschner wires, or any other variants that have a cutting tip to penetrate bone. Incision points on the soft tissue, for example, skin, are made to allow for smooth insertion of bone fasteners during subsequent procedures. For example, the incision points correspond to the positions of the apertures of the external fixator configured to receive the bone fasteners.

The fixation of the frame plate to the middle phalanx begins by drilling a through hole along a longitudinal length of a frame body. The through hole forms one of the plurality of apertures configured to receive the bone fasteners. As shown in FIG. 8b, it may form a second or third aperture 807₃ which is positioned between the two wires. A drilling system may be employed to form the through hole such that it extends from a first surface or top surface of the frame body to a second surface or bottom surface via a drilling process. Any readily available drilling system can be employed to form the through hole. For example, drilling system from Synthes or Medartis may be utilized. In one embodiment, the through hole is formed such that it is configured to form a fixed angle construct with a bone fastener such as a screw. For example, the screw is inserted into the through hole at an angle perpendicular to the longitudinal axis of the middle bone. This increases biomechanical strength of the frame plate for supporting the injured site.

An internal diameter of the through hole is configured to be compatible to receive a screw selected during the pre-operative planning. For example, the selected screw can include cortical screws, cancellous screws and locking head screws. Any other types of selected screw suitable for fastening into a bone at the injured site can also be used. In one embodiment, the internal diameter, d, of the through hole or aperture is 1.25 mm.

In one embodiment, the through hole is tapped. For example, the through hole includes grooves suitable for receiving threaded screws. As discussed, the threaded screw may include screws with threaded screw heads. For example, as shown in FIG. 8b, the threaded screw 811 is inserted into the drilled through hole of the external fixator and into a portion of the middle phalanx, for example the cortical part of the middle phalanx. The mating between the grooved aperture and the threaded screw head 813 of the screw 811 maintains the frame plate and the bone in a locked configuration without requiring bone-plate contact compression. Therefore, this allows the external fixator to maintain a gap G between the frame plate and the attached bone. The gap G allows for sufficient space for possible soft tissue swelling. Further, due to uninterrupted blood supply to the bone without any compression on periosteum, this facilitates a faster bone healing process.

Once the screw is locked in position at one of the apertures of the frame plate, the middle phalanx is considered a fixated bone. For example, the screw secures the middle phalanx to the frame plate. The process of inserting the screw into the through hole and a portion of the middle phalanx may be conducted under the supervision of an imaging equipment. This allows a better evaluation of a depth and direction of the inserted screw into the middle phalanx.

Following the insertion of one screw into the second or third aperture, the two wires 803a and 803b are subsequently replaced with screws 811 as illustrated in FIG. 8c. Fixation of the middle phalanx to the frame plate using the screws is completed in FIG. 8d. Even though it is shown that 3 screws are used in the fixation process, it is to be understood that, depending on an extent, type or position of injury, the number of screws and apertures on the frame plate can be less than or more than 3 to provide for an effective bone treatment process.

In FIG. 8e, assembling of a primary mount of the first configuration of the external fixator begins. An internal rod 821 is inserted across the epicondylar axis of a bone adjacent to the now fixated middle bone 805, for example the proximal phalanx 823. The internal rod is configured to couple the proximal phalanx to the frame plate 801 and the fixated middle phalanx. The internal rod may include any types of wire suitable for use with external fixator. In one embodiment, the wire has a diameter ranging between 1.0 to 1.1 mm. Alternatively, providing wires of other diameters may also be useful.

The wire is cut to a wire length and bent to form a cantilevered design in FIG. 8f As shown, the two ends of the wire protruding out from the skin are bent in first and second directions to form S-shaped overhangs 825₁ and 825₂. The wire length and the S-shaped overhangs facilitate minimized overall dimension of the external fixator. It is to be understood that the wire length varies along with the application of the external fixator on different users or patients.

Referring to FIG. 8g, a first external rod 831ₐ is bent to form a U-shaped rod 831_{b}. The first external rod may include any types of wire suitable for use with external fixator. In one embodiment, the wire has a length ranging between 1.25 to 1.44 mm. Alternatively, providing wires with other lengths for the external rod may also be useful. In one embodiment, the U-shaped rod includes a central limb 833 with a length of 6.8 mm. Having a central limb with other lengths may also be useful.

In FIG. 8h, the free ends 832₁₋₂ of the U-shaped rod 831_{b} is inserted into the respective first and second through holes 834 of the first and second flanges 836 of the frame plate 801. The central limb of the U-shaped rod is slotted and secured under the recess 838 which is formed on an underside surface of a first end of the frame plate, as shown in FIG. 8i. The recess 838 and the first and second through holes 834 collectively function as mounting points of the primary mount to support the first external rod. The assembly of the primary mount with the first external rod forms a primary frame.

In one embodiment, the first external rod extends beyond the frame plate 801 to increase a total length of the external fixator for bridging adjacent bones beneath the external fixator. As seen in FIG. 8j, the free ends of the first external rod 831_{b} may be cut and adjusted to a length E configured to provide a distraction at the PIPJ. Similar to the internal rod, the two free ends of the first external rod are also bent in first and second directions to form S-shaped overhangs 841.

The distraction includes an extent of rotation of a phalanx along an anatomical center of rotation at the joint. In one embodiment, the distraction is controlled by varying a length E of the external rod, for example, the lengths of the free ends. A longer length increases the connecting distance D between the coupling ends of the external rod 831_{b} and the internal rod 821, and therefore permits a wider degree of rotation about the joint.

In FIG. 8k, first connectors 842 are configured to connect the U-shaped rod 831_{b} to the internal rod 821. Various types of connectors that are elastic and flexible can be used. In one embodiment, the connectors are dental rubber bands. In one embodiment, as shown, the first connectors connect the extended free ends 831₁₋₂ of the U-shaped rod to the free ends 825₁₋₂ of the internal rod at both sides, for example left and right sides, of the injured side to form a bilateral primary frame. Alternatively, a unilateral primary frame may also be provided. For example, the first connector connects one side of the U-shaped rod to the same corresponding side of the internal rod. In this case, the injured side may be used to determine the side which will be connected to form the unilateral primary frame.

As discussed, a primary frame forms the first configuration of the external fixator. The first configuration is configured to provide load support and joint mobility at an injured site. In another embodiment, when the injured site includes a fractured and/or dislocated joint, the primary frame of the first configuration also controls a first force vector for joint alignment and fracture reduction.

In cases where the first configuration is not sufficient to provide smooth joint mobility and/or complete fracture reduction, the secondary frame can be assembled to form a second configuration. In FIG. 8l, the assembly of the secondary frame begins with engaging a second external rod 844 with a secondary mount at the collar head member 848. As shown, the second external rod is inserted through the third through hole 846 of the secondary mount. In one embodiment, the second external rod is a wire having a length ranging between 1.0 to 1.1 mm. Providing other types of wire having other lengths may also be useful. Similar to the internal rod, two free ends of the second external rod are also cut and then bent in first and second directions to form S-shaped overhangs.

In one embodiment, as shown in FIG. 8m, second connectors 852 connect both extended free ends 851₁₋₂ of the second external rod 844 to the free ends 825₁₋₂ of the internal rod 821 at both sides, for example left and right sides, of the injured side. This forms a bilateral secondary frame. The second configuration can include bilateral primary and secondary frames. Alternatively, in the case of a unilateral injured site, for example, either at the right or left side, a second configuration having unilateral primary and secondary frames may be provided. Various types of connectors that are elastic and flexible can be used. In one embodiment, the connectors are dental rubber bands.

As discussed, the primary and secondary frames of the second configuration respectively exerts first and second force vectors F₁ and F₂. As a result of a summation of the first and second force vectors, a net displacement R in a desired direction is generated. This achieves a better fracture reduction result for proper joint realignment or smoother joint mobility.

The inventive concept of the present disclosure may be embodied in other specific forms without departing from the spirit or essential characteristics thereof. The foregoing embodiments, therefore, are to be considered in all respects illustrative rather than limiting the invention described herein. Scope of the invention is thus indicated by the appended claims, rather than by the foregoing description, and all changes that come within the meaning and range of equivalency of the claims are intended to be embraced therein.

What is claimed is:

1. An external fixator comprising:
an elongated frame plate having a frame body with first and second sides and top and bottom surfaces;
apertures disposed along a length of the frame body, the apertures extend through the frame body from the top surface to the bottom surface, the apertures are configured to accommodate bone fasteners to secure the elongated frame plate;
a primary mount configured to accommodate a primary rod which, when mounted onto the elongated frame member, extends beyond a second end of the frame plate; and
a secondary mount configured to accommodate a secondary rod, the secondary mount is disposed at about a first end of the frame body, the secondary rod, when mounted, extends transversely across the sides of the frame body.

2. A method for supporting an injured joint comprising:
coupling an elongated frame plate of a fixator system to a bone to form a fixated bone using bone fasteners through apertures on the elongated frame plate, wherein the frame plate includes primary and secondary mounts configured to respectively accommodate primary and secondary rods;
mounting the primary rod to the primary mount of the frame plate, the primary rod extends beyond a second end of the elongated frame plate;
inserting an internal rod, along a transverse plane, into an adjacent bone next to the fixated bone, wherein the primary rod and the internal rod forms a force vectoring subsystem of the fixator system; and
coupling the internal rod to the primary rod by at least one vectoring connector to a cantilevered frame, wherein the cantilevered frame is configured to minimize interference while enabling joint mobilization.
